(19) United States
(12) Plant Patent Application Publication
(10) Pub. No.: US 2007/0300344 P1
(43) Pub. Date: Dec. 27, 2007

(54) Lupinus plant named 'Persian Slipper'

(76) Inventor: Sarah Frances Conibear, Devon (GB)

Correspondence Address: THE WEBB LAW FIRM, P.C., 700 KOPPERS BUILDING, 436 SEVENTH AVENUE, PITTSBURGH, PA, 15219, US

(21) Appl. No.: 11/474,101

(22) Filed: Jun. 23, 2006

(51) Int. Cl. A01H 5/00

(52) U.S. Cl. PLT/263

(57) ABSTRACT

‘Persian Slipper’ is a new variety of Lupinus hybrid that exhibits numerous and large-shaped violet-colored flowers.

### BOTANICAL CLASSIFICATION

Lupinus hybrid

### VARIETAL DENOMINATION

‘Persian Slipper’

### BACKGROUND OF THE INVENTION

The present invention comprises a new and distinct cultivar of Lupinus hybrid known by the varietal name ‘Persian Slipper’. The new variety was developed in Great Britain. The new variety is a result of cross breeding Russell Hybrid Lupinus Seedling #WCL 066/96 (female parent) (unpatented) and an unknown Lupinus male parent (unpatented). The purpose of the breeding program was to develop a cultivar that exhibited improved color, habit, and bloom form from other Lupinus varieties known to the breeder. The new variety is similar in growth habit to its female parent, but differs in flower color. The new variety is similar in flower color to Lupinus hybrid ‘The Governor’, but has a more vigorous growth habit, larger flower bells, and more closely packed flower bells. The new variety was first asexually reproduced by basal cuttings/tissue culture in Great Britain. The new variety has been trial and field tested and has been found to retain its distinctive characteristics and remain true to type through successive asexual propagations. Characteristics that distinguish the new variety from others known to the breeder include:
Clear and intense flower color;
Flower form—the individual bells are packed closely on the flower spike;
Large individual bells; and
Vigorous growth habit.

The new variety was grown in open soil with additional organic fertilizer applied. The new variety initiates and develops roots in 21 days at 15° C. A finished and flowering plant is produced in 36 weeks in a 21 cm container. The new variety exhibits sparse, but thick roots, that exhibit some branching.

### DESCRIPTION OF THE DRAWINGS

The accompanying photographic drawings illustrate the new variety, with the color being as nearly true as is possible with color illustrations of this type:

FIG. 1 is a photographic image of multiple plants of the new variety; and

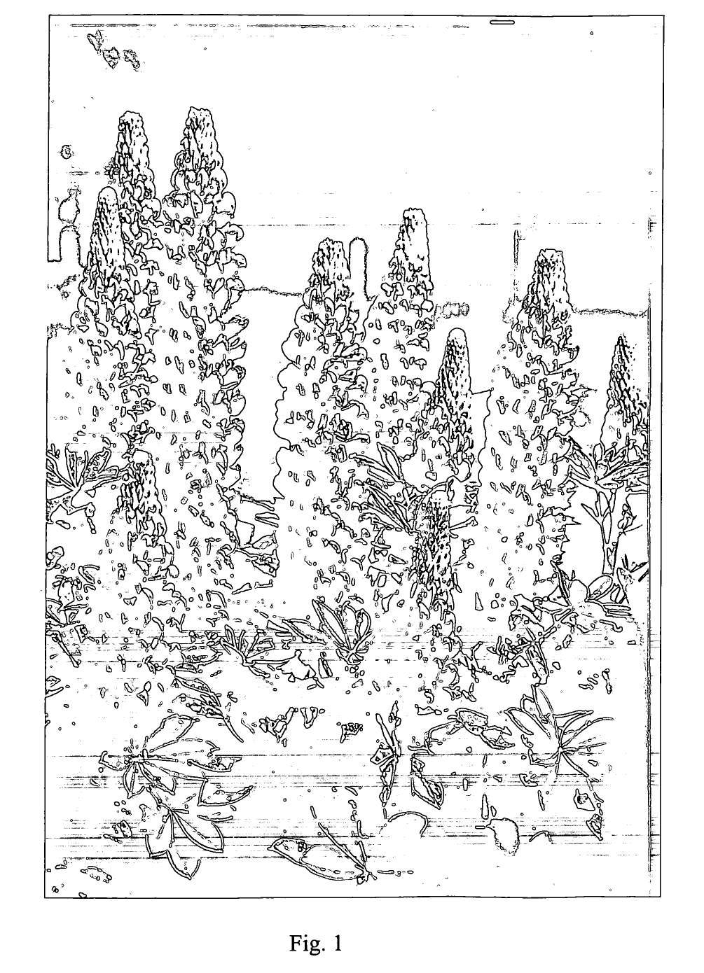

FIG. 2 is a close-up photograph of the petals of the new variety.

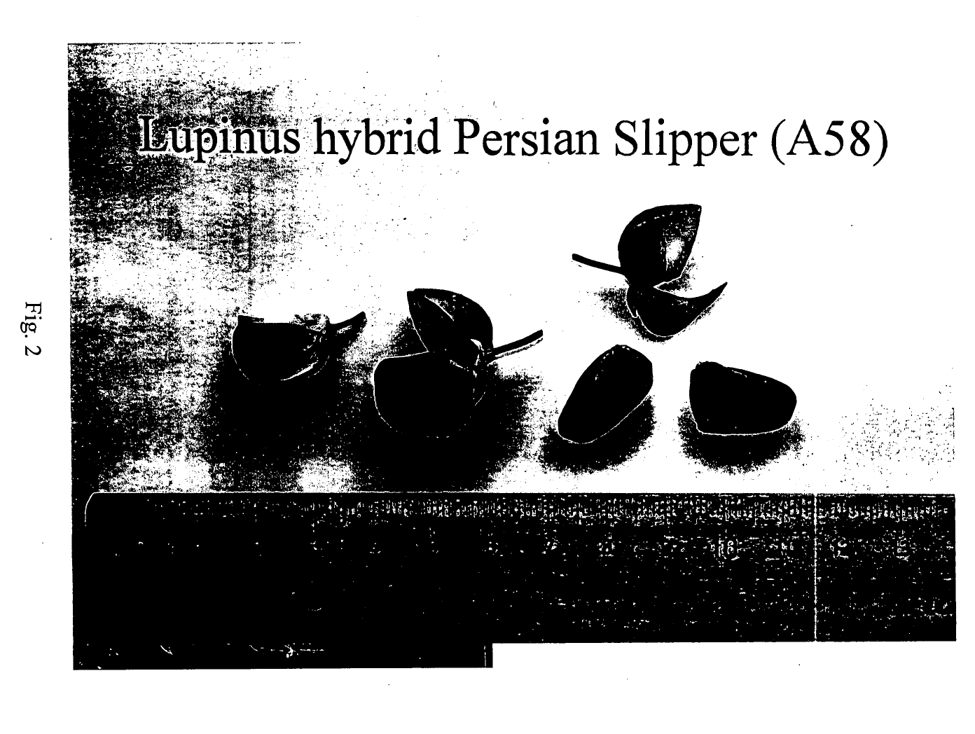

### DESCRIPTION OF THE PLANT

The following detailed description sets forth the characteristics of the new cultivar. The data which defines these characteristics were collected by asexual reproductions carried out in Great Britain. The color readings were taken outside under natural daylight. Color references are primarily to the R.H.S. Colour Chart of The Royal Horticultural Society of London.

### PLANT

Form: Mounded and symmetrical.
Stem:
   Diameter.—15 mm.
   Color.—144A/B and flushed with 59 B/C.
   Texture.—Slightly hirsute, smooth with longitudinal ridges.
   Pubescence.—Slight to medium.
Height: 70 cm.
Vigor: Good.
Branching habit: Free.
Lateral branches:
   Diameter.—10-12 mm.
   Internode length.—3.5 cm.
   Texture.—Lightly hirsute, smooth with longitudinal ridges.
   Pubescence.—Present.
   Color.—144A.
Foliage:
   Arrangement.—Ascending spiral.
   Length of leaf stalk.—Basal foliage: 28 cm. Stem foliage: 17 cm.
   Size of leaf (overall).—Length: 15.5 cm. Width: 15.5 cm.
   Leaflets.—Shape of leaflet: Oblanceolate. Number: 14. Width: 2.6 cm Length: 8.0 cm. Shape of apex: Obtuse with a point. Shape of base: Cuneate. Texture: Smooth and velvety. Aspect: Horizontal presentation. Margin type: Smooth; entire. Pubescence: Upper surface: None present. Lower surface: Slight. Color: Young leaflet: Upper surface: 137B. Lower surface: 137C. Mature leaflet: Upper surface: 137A. Lower surface: 137B. Petiole: Shape: Flattened and round. Length: Basal leaf: 28 cm. Stem leaf: 17 cm. Diameter: 5 mm. Color: 144A/B. Venation: Central; mid-rib.

### FLOWER

Bud (described when buds where just beginning to open):
   Shape.—Napiform.
   Diameter.—11 mm.
   Length.—18 mm.
   Color.—96C and suffused with 78B/C.
Natural flowering season: May through July in The Netherlands.
Flower type and habit: Spike.
   Number of flowers per spike.—193.
   Length of spike.—42 cm.
   Diameter of spike.—10.5 cm.
Corolla:
   Form.—Zygomorphic.
   Petals.—Standard Petals: Number: 2 — fused. Length: 16 mm. Width: 11 mm. Shape: Ovate. Aspect: Parallel to spike. Margin: Smooth and entire. Wing Petals: Number: 2. Length: 19 mm. Width: 11 mm. Shape: Ovoid to kidney-shaped. Aspect: Slightly tilted upward. Margin: Smooth and entire. Keel Petals: Number: 2 — fused. Length: 16 mm. Width: 8 mm. Shape: Lingulate. Aspect: Located within wing petals and pointing upwards. Margin: Smooth and entire. Texture and appearance: Smooth and satin-like. Color: When opening: Upper surface: 83C, flushed with 83A/B. Lower surface: 85B, flushed with 83D. Fully opened: Upper surface: 83B with an overlay of 83A. Lower surface: 83D.
   Throat.—Color: 4D, flushed with 4B. Texture: Smooth and satin-like.
Calyx:
   Form.—Two unequal halves.
   Length.—6 mm.
   Diameter.—4 mm.
   Sepal shape.—Elliptic.
   Sepal margin.—Entire.
   Sepal texture.—Smooth with slight pubescence.
   Sepal size.—Length: 4 mm. Width: 2.5 mm.
   Sepal color.—Upper surface: 85D, flushed with 85C. Lower surface: 85D, flushed with 85C.
Fragrance: Slight.
Disease resistance/susceptibility: Susceptible to powdery mildew.
Temperature tolerance: Medium to good.
Lasting quality: Medium to good.

### REPRODUCTIVE ORGANS

Stamens:
   Number (per flower).—There are two groups. A first group with 6 stamens and a second group with 4 stamens.
   Filaments.—Length: First group: 4 mm. Second group: 11 mm. Fused: Yes.
   Anthers.—Shape: Pencil-like. Length: 2 mm. Color: 31A.
   Pollen.—Color: 31A. Amount: Plentiful.
Pistils:
   Length.—13 mm.
   Style.—Length: 13 mm. Color: 150C.
   Stigma.—Shape: Spherical. Color: 150C.
Seed/fruit production:
   Description of conditions when produced.—Produced freely.
   Color.—165A.
   Size.—1.0 mm.
   Number.—Approximately 2000 per plant.

I claim:

1. A new and distinct variety of Lupinus hybrid plant named ‘Persian Slipper’ as is herein described and illustrated.
